(19) United States
(12) Patent Application Publication
(10) Pub. No.: US 2011/0048433 A1
(43) Pub. Date: Mar. 3, 2011

(54) Method for forming an interventional aid with the aid of self-organizing nanorobots consisting of catoms and associated system unit

(76) Inventor: Marcus Pfister, Bubenreuth (DE)

(21) Appl. No.: 12/872,015

(22) Filed: Aug. 31, 2010

(30) Foreign Application Priority Data
Aug. 31, 2009 (DE) 10 2009 039 520.2
Dec. 22, 2009 (DE) 10 2009 060 092.2
Feb. 24, 2010 (DE) 10 2010 009 017.4

(51) Int. Cl. A61B 19/00

(52) U.S. Cl. 128/898; 128/899; 977/904

(57) ABSTRACT

A method for forming at least a part of a preferably endovascular interventional aid with the aid of self-organizing nanorobots consisting of catoms and an associated system are provided. A form of the required interventional aid is determined from at least one 3D image data record of a target region. The determined form is converted to a readable and executable program code for the respective catoms of the nanorobots and is transferred to a storage unit. The program code is executed which prompts self-organization of the previously unstructured catoms to form the required interventional aid according to the previously determined form.

### CROSS REFERENCE TO RELATED APPLICATIONS

This application claims priority of German application No. 10 2009 039 520.2 filed Aug. 31, 2009, German application No. 10 2009 060 092.2 filed Dec. 22, 2009, and German application No. 10 2010 009 014.4 filed Feb. 24, 2010, which are incorporated by reference herein in its entirety.

### FIELD OF THE INVENTION

The invention relates to a method for forming at least a part of a preferably endovascular interventional aid with the aid of self-organizing nanorobots consisting of catoms and an associated system unit.

### BACKGROUND TO THE INVENTION

Very many examinations and interventions on patients are carried out in a minimally invasive manner. With such procedures instruments (catheters, etc.) are inserted into the patient through small openings (e.g. access in the groin) to carry out examinations or therapies in the heart, head or abdomen. These procedures are monitored with the aid of two-dimensional x-ray fluoroscopy images, e.g. by means of C-arm angiography systems. Modem angiography systems are also able to record three-dimensional images of the examination region by rotating the C-arm about the patient and reconstructing the rotation sequences.

With many of these interventions “therapeutic aids” are introduced into the patient, e.g.

stents, e.g. to assist with the widening of narrowed vessels or to repair vessel segments,

flexible coils, e.g. to close off aneurysms,

microbeads, e.g. to close off/embolize vessels supplying a tumor.

There are in principle three problems:

1. To achieve optimum therapeutic success, such aids generally have to be tailored very precisely to the respective anatomy of the patient. The corresponding selection requires time and very precise measuring of the anatomy, e.g. based on a 3D data record. Such measurements are generally taken either manually or with the aid of 3D image processing (e.g. segmentation of the vessel segment). Also a 100% suitable aid (e.g. a precisely fitting stent) is often not available and must be produced specially or the “second best” must be selected.

2. The selected aid, e.g. coil or stent, must be positioned optimally. For example stents must not close off any outgoing vessels and coils must not project into the carrier vessel (to avoid embolisms). The latter is particularly problematic, as coils can assume different shapes from those planned on introduction into an aneurysm.

2. Navigation of the guide wire or catheter to the site to be treated. During the treatment of cerebral aneurysms for example very small or narrow vessel branches have to be treated. In the case of treatments in the heart in contrast certain points have to be approached precisely in the organ with its movement due to the heartbeat.

The background to the present invention is the forming and introduction of such aids or of navigation aids, by what is known as Dynamic Physical Rendering (DPR) or Claytronics [1,2,3,4,5], the name coming from the eponymous interdisciplinary Claytronics researcher group at Carnegie Mellon University. The research subject (a current research sub-field of nanotechnology in convergence with robotics) is also referred to as programmable (or intelligent) material. The object of the research field is to organize “intelligent” autonomous “material particles” in other words autonomous nanorobots, by means of what is known as Dynamic Physical Rendering (DPR) to form actually existing macrobodies of any programmable form. The specific nanorobots used in Claytronics are known as catoms, combining the terms Claytronics and atom. These are in principle small, autonomous robots, which are able to self-organize to assume a previously commonly programmed larger configuration.

Intelligent nanorobots are known as a collective from [5]. They can act independently, introduce drugs into cells, etc. Ways are described in which the CNRs (Collective of Nanorobots) can be moved into position. They are for example bound to antibodies, which draw the CNRs approximately for example into inflamed regions. They are also positioned on stents that have been introduced in the conventional manner and then serve as a type of base. From there they carry out their tasks (e.g. combating stroke or inflammation) and then return to it.

Publications, e.g. [1,3,4] show that the localization and self-organization of the robots can already be applied for small units or in simulations. Challenges for endovascular application are still the size and energy supply of the units to be used. However the miniaturization of such units is constantly advancing rapidly.

### SUMMARY OF THE INVENTION

The invention is based on the task of making it possible to use “intelligent” autonomous “material particles”, in other words the abovementioned autonomous nanorobots for minimally invasive procedures by means of what is known as Dynamic Physical Rendering (DPR).

The object is achieved with the method and apparatus according to the independent claims. Advantageous embodiments of the method and apparatus are set out in the dependent claims or can be derived from the description which follows and the exemplary embodiments.

One aspect of the invention is a method for forming at least a part of an interventional aid with the aid of self-organizing nanorobots consisting of catoms, having the following steps:
Using at least one 3D image data record of a target region, preferably of the region to be treated,
Determining a form of the required part(s) of the interventional aid from the at least one 3D image data record,
Converting the determined form to a readable and executable program code for the respective catoms of the nanorobots and transferring it to these or its storage unit there,
Activating the execution of the program code, which prompts self-organization of the previously unstructured catoms to form the required interventional aid according to the previously determined form.

A further aspect of the invention is a system unit or apparatus for organizing nanorobots consisting of catoms, which are suitable for implementing the method, comprising:
a number of nanorobots, with each nanorobot comprising at least a part of a program code, by means of which the nanorobots are configured to form at least a part of the interventional aid with the aid of the nanorobots by communicating and exchanging information with other nanorobots,
wherein the nanorobots can be or have been introduced into a target region, preferably of the region to be treated,
wherein the nanorobots have means for executing the program code, which can be activated by prompting self-organization of the previously unstructured catoms to form the at least one part of the required interventional aid according to the previously determined form.

The at least one part of the required interventional aid can preferably be used in an endovascular target region or for the purpose of navigation in such a target region. The at least one part of the required interventional aid can be represented by an interventional aid that is complete and/or remains stationary in the body, preferably at least one stent, coil, or by a temporary aid, in particular one formed momentarily just for navigation purposes, such as a catheter and/or guide wire, or by preferably at least a part of at least one catheter and/or catheter tip and/or at least one guide wire and/or at least one guidance aid.

In one advantageous development of the invention provision is made for it to be possible to introduce the nanorobots into the target region with the aid of a catheter.

A timer or position sensor can be used to trigger the activation of the program code.

The region to be treated can advantageously be segmented from the 3D image data record, segmentation being carried out based on set marking points.

The specific form of the required interventional aid can be based on a three-dimensional model, which demonstrates variations of a specified basic form (e.g. of a cylinder).

It is thus possible to introduce and position the aids quickly, automatically, optimally and in a specific manner for each patient without prior manual measuring or dimensioning.

### BRIEF DESCRIPTION OF THE DRAWINGS

Further advantages, details and developments of the invention will emerge from the description which follows of exemplary embodiments in conjunction with the drawings, in which:

FIGS. 1a and 1b show catoms, in other words the Claytronics hardware, the diameter of which can be below 1 mm for example,

FIGS. 2a, 2b and 2c show an example of the forming of a stationary aid, specifically a stent in an abdominal aneurysm,

FIGS. 3a, 3b and 3c show an example of the forming of a stationary aid, specifically a coil in a cerebral aneurysm,

FIGS. 4a, 4b and 4c show an example of a stationary aid for embolizing a liver tumor,

FIGS. 5a, 5b, 5c, 5d, 6a, 6b, 6c, 6d and 7a, 7b, 7c, 7d show an example of a temporary aid for navigation in difficult “branches” in brain vessels and

FIGS. 8a, 8b, 8c, 8d, 9a, 9b, 9c, 9d and 10a, 10b, 10c, 10d show an example of a temporary aid for navigation for the purpose of ablation in cases of cardiac arrhythmia.

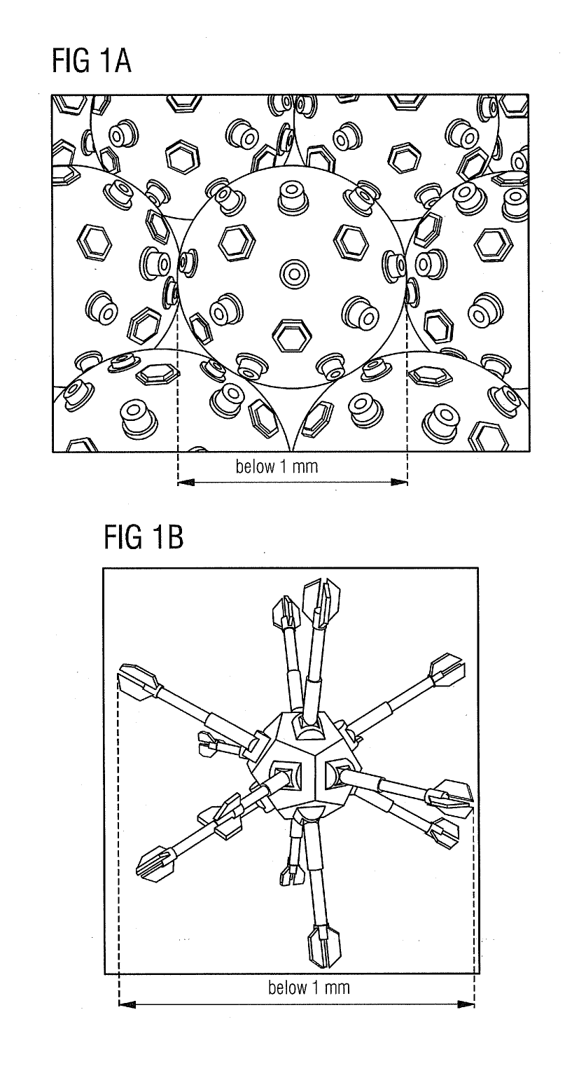

### DETAILED DESCRIPTION OF THE INVENTION

The inventive application for endovascular interventions now provides for the temporary or permanent “cloning” of previously determined anatomical structures, to facilitate the selection and positioning of stents and coils for example.

The forming of a stationary interventional aid from catoms is described below with reference to three examples:

The starting point in each instance is a 3D data record (e.g. a CT angiography, a rotational angiography or a C-arm CT) of the region to be treated. In some circumstances segmentation (e.g. vessel segmentation) of the data record may be advantageous.

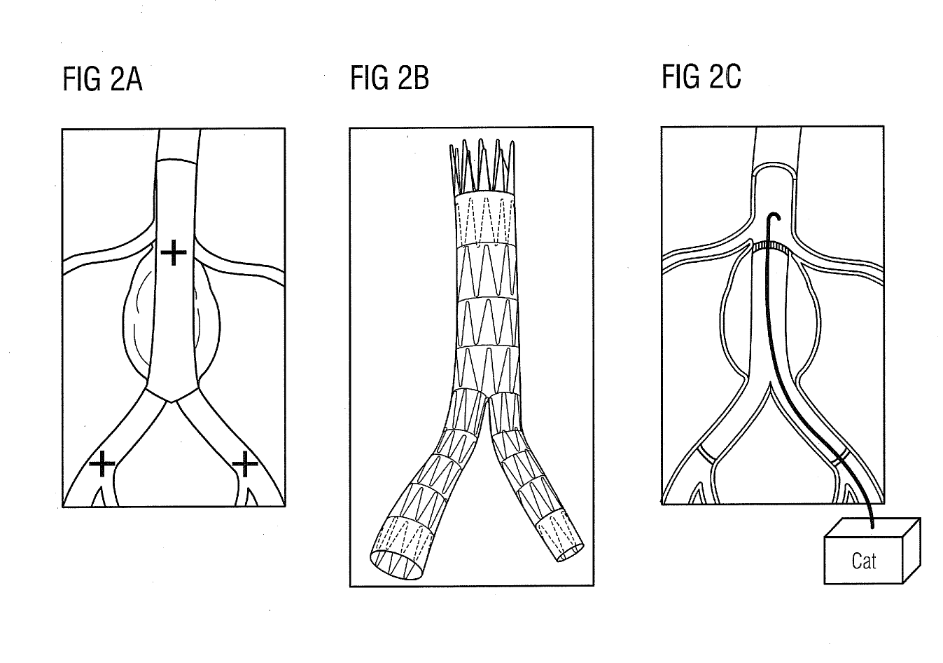

FIGS. 2a, 2b and 2c show an example of the forming of a stent in an abdominal aneurysm.

The starting point is a 3D data record (e.g. a CT angiography) of the aneurysm. Based on a segmentation of the data record (e.g. into lumen and thrombus) and any marking or measurement points set by the user (marking the planned stent limits for example), as marked by means of crosses in FIG. 2a, it is possible to calculate the form of the “optimum” stent automatically, e.g. as a grid model, as shown for example in FIG. 2b. This model is then the programming for the catoms Cat, which can be introduced for example by way of a catheter in unstructured form into the aneurysm (see FIG. 2c). The units organize themselves in situ, as set out above (see FIG. 2b), to form the stent of previously determined form (see FIG. 2c).

Other advantages, apart from the optimum tailoring of the stent to the vessel, are optimum positioning (without the risk of closing off outgoing vessels, e.g. renal arteries) and uncomplicated introduction compared with the conventional positioning of an abdominal stent.

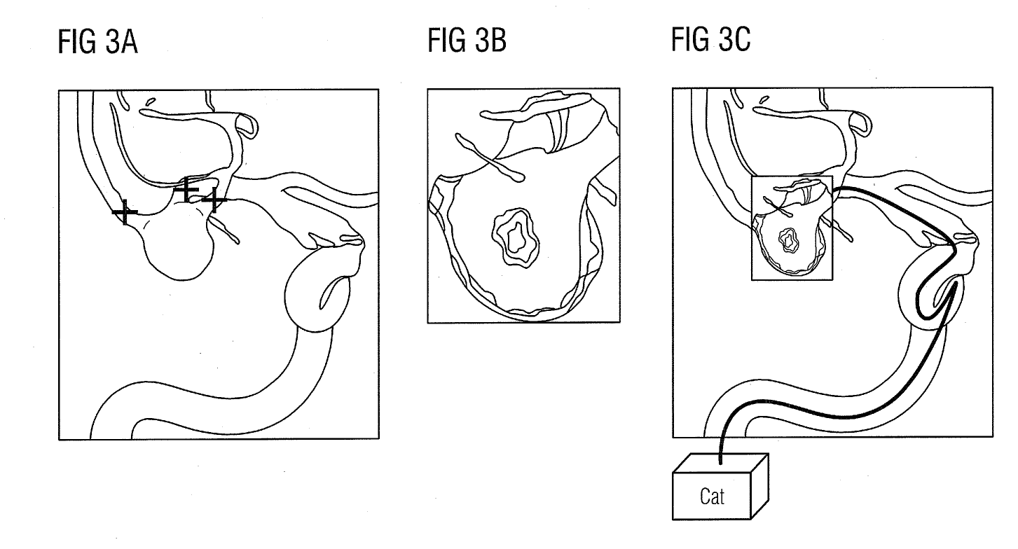

FIGS. 3a, 3b and 3c show an example of the forming of a coil in a cerebral aneurysm.

The starting point is a 3D data record (e.g. a CT or rotational angiography) of the aneurysm. Based on a segmentation of the data record and any measurement points set by the user, which designate the limits of the region to be closed off here for example and are shown marked by crosses in FIG. 3a, it is possible to calculate the form of the “optimum” coil automatically, e.g. as a grid model, as shown for example in FIG. 3b. In addition to the coil for closing off the aneurysm, it is also possible to take into account any plastic vessel molds for modeling the vessels in question. It is possible to plan a very complex “repair aid” in this manner. This model is then the programming for the catoms Cat, which can be introduced for example by way of a catheter into the aneurysm (see FIG. 3c). The units organize themselves in situ, as set out above (see FIG. 3b), to form the coil or stent of previously determined form (see FIG. 3c).

Other advantages, apart from the optimum design of the coil, are optimum positioning (without the risk of closing off or otherwise impairing the carrier vessel) and uncomplicated introduction compared with standard coils and stents for an intracranial aneurysm.

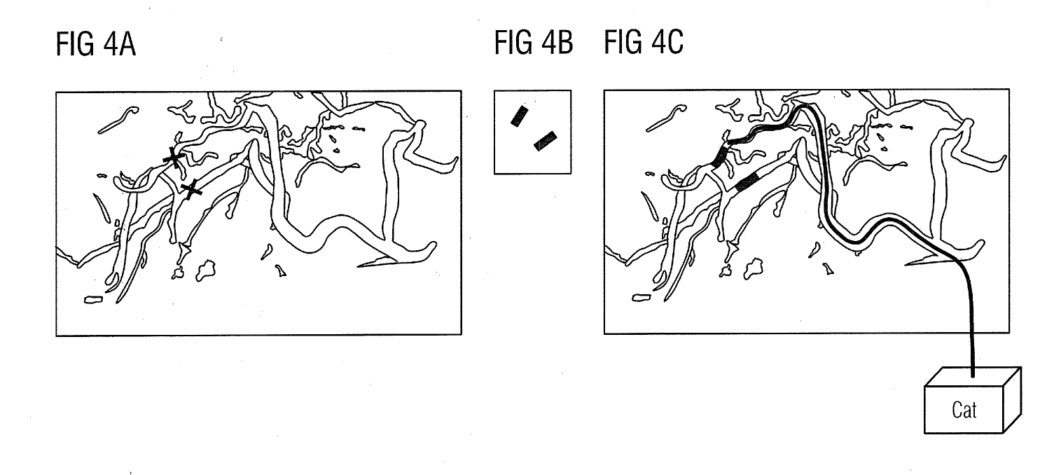

FIGS. 4a, 4b and 4c show an example based on the embolization of a liver tumor.

The starting point is a 3D data record (e.g. a CT or rotational angiography) of the liver vessel supplying the tumor. Based on a segmentation of the data record and any measurement points set by the user (here marking the vessels to be closed off for example), which are marked by crosses in FIG. 4a, it is possible to calculate the form of the “optimum” blocks for the arteries to be embolized, e.g. as a grid model, as shown for example in FIG. 4b. This model is then the programming for the catoms Cat, which can be introduced for example by way of a catheter into the liver arteries (see FIG. 4c). The units organize themselves in situ, as set out above (see FIG. 4b), to form the embolizers of previously determined form (see FIG. 4c).

The described measurement points can also be proposed automatically by the segmentation for the respective application. It is thus possible for example for the stent limits for an abdominal stent (see FIG. 2a) to be proposed automatically by the location of the renal artery exits and the branches of the leg arteries. For an intracranial aneurysm the branches of the supplying vessels could be proposed as measurement points for example.

In a further embodiment the form of the interventional aid, e.g. a stent, cannot be determined based on the 3D model but it can be selected from a selection of available or predetermined, in some instances geometric, standard forms, e.g. stents of different lengths and diameters. The catoms then assume the corresponding form. This also has the advantage of simple introduction of the aid.

The forming of non-stationary or temporary interventional aids, in particular for the purpose of navigation, from catoms is described below with reference to two examples:

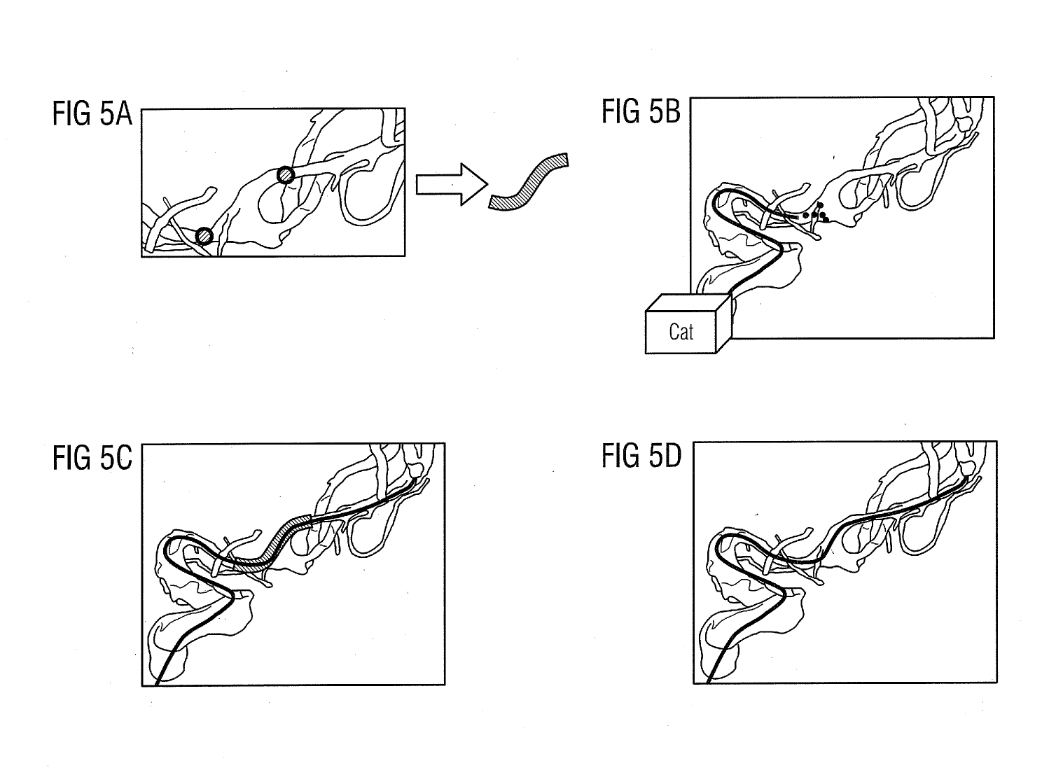

FIGS. 5a, 5b, 5c, 5d, 6a, 6b, 6c, 6d and 7a, 7b, 7c, 7d show an example of navigation in difficult “branches” in brain vessels. The problem with treating for example cerebral aneurysms is the navigation of the guide wire or catheter to the site to be treated. Cerebral vessel systems in particular contain narrow turns or complex branches, which cannot be passed through easily. The following three options are possible for this example:

1. Forming guides according to FIGS. 5a, 5b, 5c, 5d: starting point is a 3D data record (e.g. a CT or rotational angiography) of the brain vessels. Based on a segmentation of the data record and any measurement points set by the user, the physician is able to determine the path to be followed for potentially difficult to navigate “branches” (see FIG. 5a). The form of the “catheter guide” to be assumed later by the catoms is therefore determined based on the 3D data record. When the physician reaches the branch with a catheter, the catoms Cat are first introduced unstructured (see FIG. 5b). They then form themselves into the required “guide” in situ (see FIG. 5c), so that the catheter can be pushed easily and quickly through the branch in the correct direction. Once the branch has been passed through successfully, the fowl can break down again (see FIG. 5d). The catoms can then be “extracted” for example or can remain unstructured in the body.

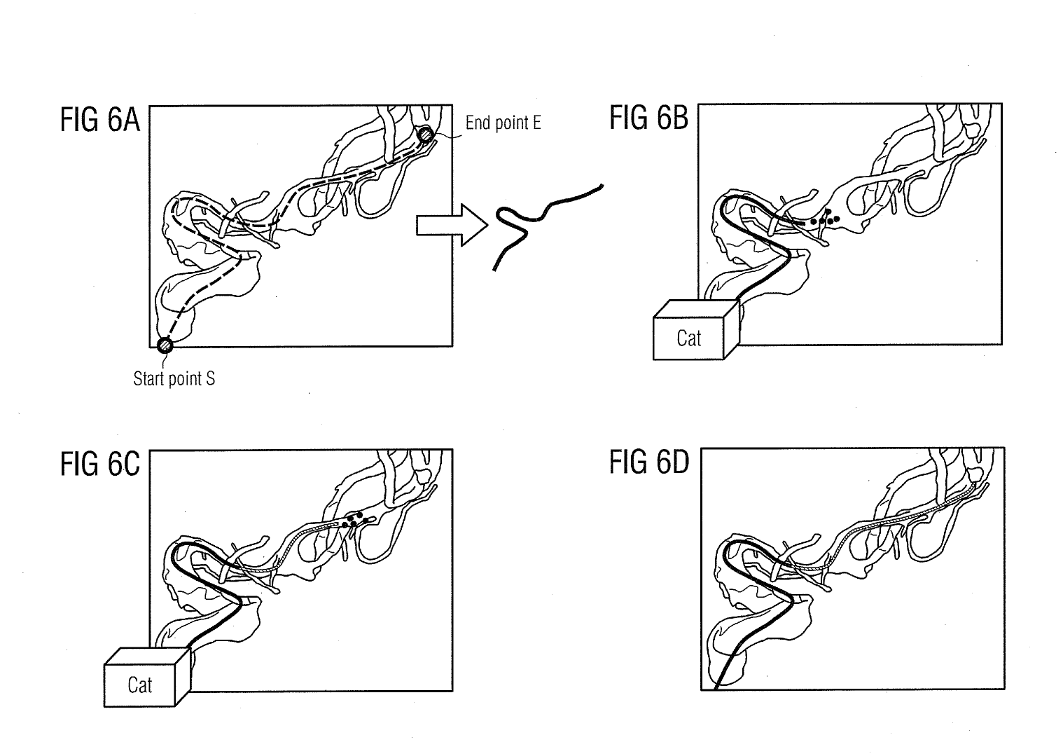

2. Forming a complete instrument (see FIGS. 6a, 6b, 6c, 6d): starting point is a 3D data record (e.g. a CT or rotational angiography) of the brain vessels. The user now simply marks the end point E of the navigation, e.g. the aneurysm, and optionally one point in an easy to reach part of the supplying artery (see FIG. 6a). Based on a segmentation of the data record the system calculates the best connecting line to the navigation target. This is the form of the catheter to be formed later by the catoms Cat. When the physician reaches a point close to the selected start point S, catoms are introduced gradually (in unstructured form) (see FIG. 6b). These then form themselves in situ into a thin tube that follows the previously defined form, thereby gradually forming the catheter (see FIG. 6c) until the target region is reached (see FIG. 6d). After the end of the intervention the form can break down again. The catoms can then be “extracted” for example or can remain unstructured in the body.

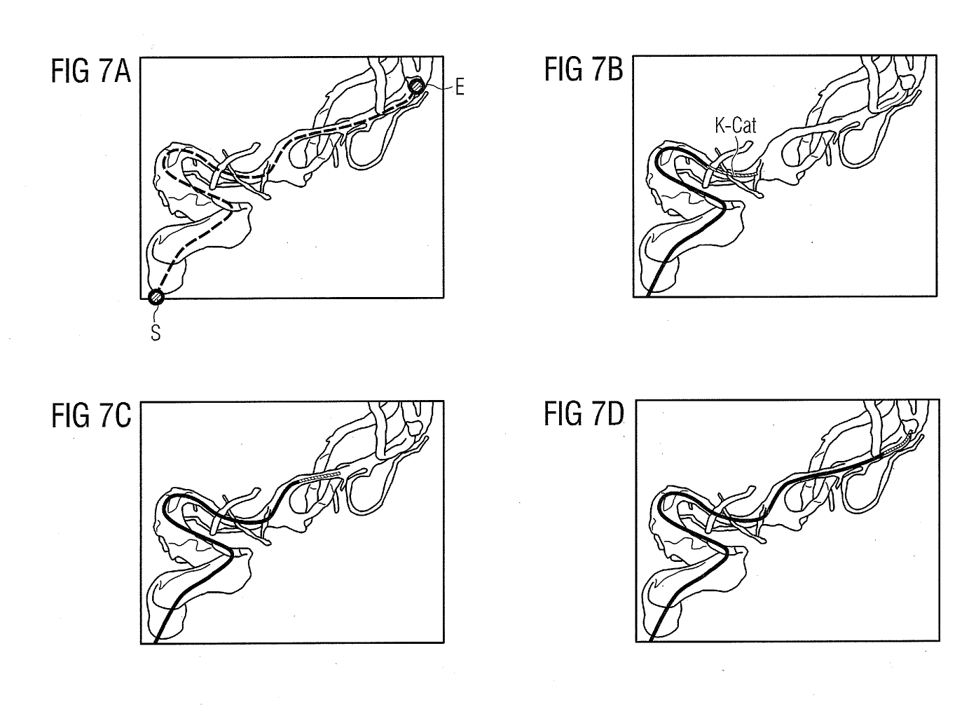

3. Forming parts of an instrument (see FIGS. 7a, 7b, 7c, 7d): starting point is a 3D data record (e.g. a CT or rotational angiography) of the brain vessels. The user simply marks the end point of the navigation, e.g. the aneurysm, and optionally one point in an easy to reach part of the supplying artery (FIG. 7a). Based on a segmentation of the data record the system calculates the best connecting line to the navigation target. The physician now introduces a catheter K_Cat, the catheter tip of which is formed from catoms and can be localized (e.g. by means of a position sensor). When the physician reaches a point beyond the selected start point S, the hitherto unstructured catoms automatically form themselves into “bends”, which correspond principally to the curves and branches of the calculated connecting line (FIGS. 7b, 7c, 7d). The instrument is thus formed optimally for each branch to facilitate navigation. The catoms then do not have to be “extracted” or remain unstructured in the body, as they are a fixed part of the instrument used.

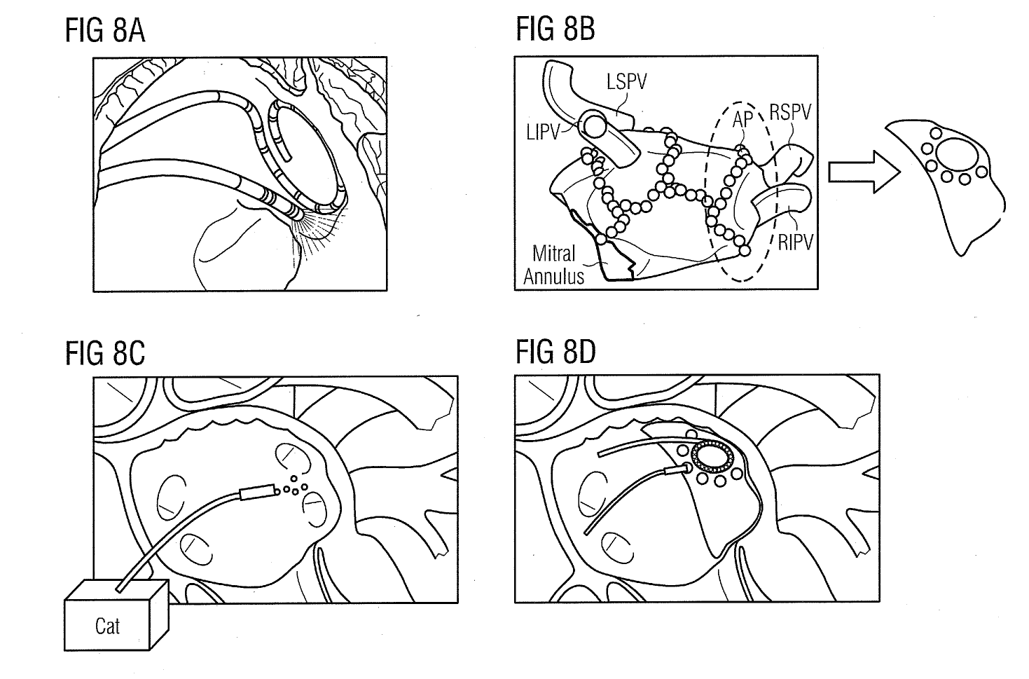

FIGS. 8a, 8b, 8c, 8d, 9a, 9b, 9c, 9d and 10a, 10b, 10c, 10d show an example of navigation for the purpose of ablation in cases of cardiac arrhythmia. In this procedure certain nerve paths are obliterated (generally electrothermally) in the auricles of the heart, to prevent unwanted impulse conduction. What is known as a “lasso catheter” is introduced here in the manner of an electrode, into one of the pulmonary veins, in order then to carry out the obliteration with an electric ablation catheter. The problem here is the precise approach to the correct points, so that on the one hand the treatment is successful and on the other hand greater damage is not caused. The procedure is made more difficult by heart movement, which makes it difficult to approach the points precisely. The following three options are possible for this example:

1. Forming guides (see FIGS. 8a, 8b, 8c, 8d): Certain nerve paths are obliterated (generally electrothermally) in the ventricles, to prevent unwanted impulse conduction (see FIG. 8a). Starting point is a 3D data record (e.g. a CT or MR) of the corresponding ventricle. The physician can mark the ablation points AP to be approached later (see FIG. 8b) in a segmentation of the data record. The form of the “catheter guide” to be assumed later by the catoms is determined based on the 3D data record and the points planned therein. In this instance these should form a “mask” covering the ventricle wall and only exposing the points to be obliterated (in some instances with a corresponding guide (see FIG. 8b)). The physician can now introduce the catoms unstructured (FIG. 8c), these then forming themselves into the desired “mask” in situ (FIG. 8d), so that the ablation catheter can be moved easily and quickly to the corresponding points while the remainder of the heart wall is protected. Once the points have been successfully reached, the farm can break down again. After the end of the intervention the form can break down again. The catoms can then be “extracted” for example or can remain unstructured in the body.

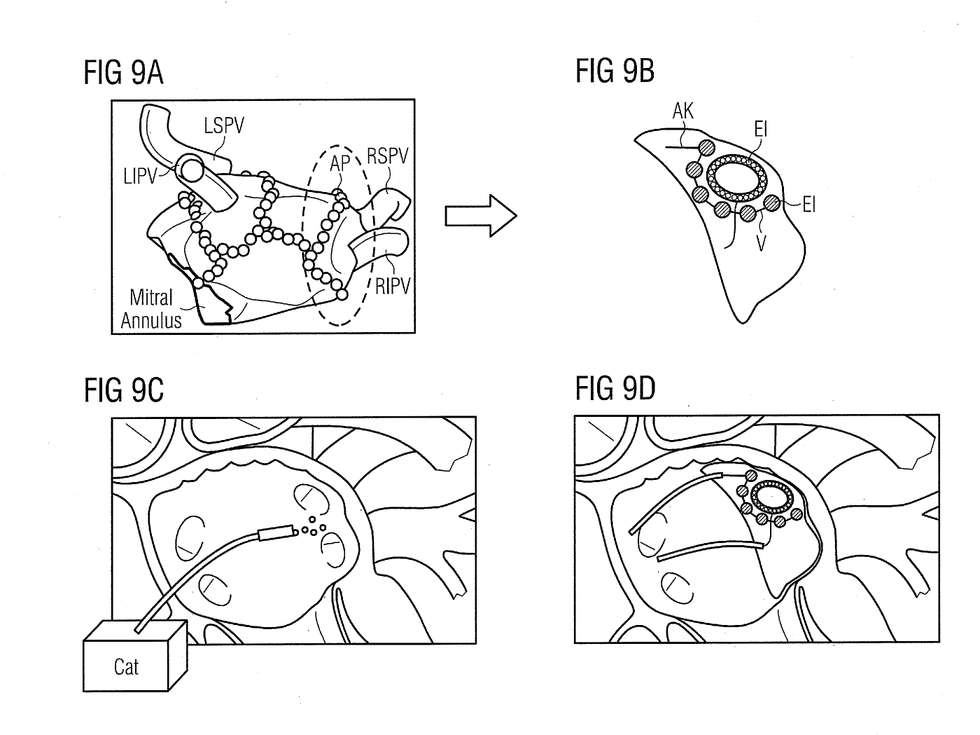

2. Forming a complete instrument (FIGS. 9a, 9b, 9c, 9d): starting point is a 3D data record (e.g. a CT or MR) of the corresponding ventricle. The physician can mark the ablation points AP to be approached later (see FIG. 9a) in a segmentation of the data record. The form of the ablation instrument to be formed later by the catoms is determined based on the 3D data record and the points planned therein. In this instance these can form a “mask”, which covers the ventricle wall and configures conductive electrodes E1 connected together by connections V at the points to be obliterated (see FIG. 9b). A lasso catheter can also be formed in some instances. Simple terminals can also be formed, on which the current-carrying catheters are positioned. The physician can now introduce the catoms unstructured (see FIG. 9c), these forming themselves into the required instrument in situ (FIG. 9d). The terminals AK now only have to be “populated” from outside with the electric catheters, so that ablation can take place safely and in one step despite heart movement. Once ablation has been completed successfully, the form can break down again. The catoms can then be “extracted” for example or can remain unstructured in the body.

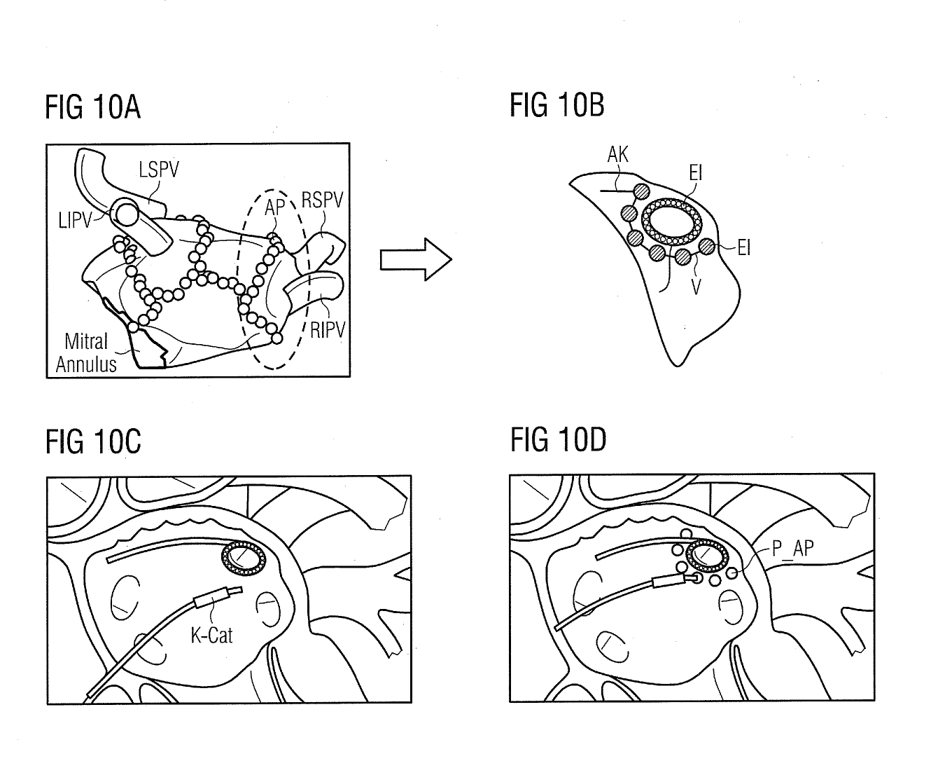

3. Forming parts of an instrument for the purpose of navigation (FIGS. 10a, 10b, 10c, 10d): starting point is a 3D data record (e.g. a CT or MR) of the corresponding ventricle. The physician can mark the ablation points AP to be approached later in a segmentation of the data record (FIG. 10a). Their 3D position in space is therefore known. These can form a “mask” which covers the ventricle wall and configures conductive electrodes E1 connected together by connections V at the points to be obliterated (see FIG. 10b). A lasso catheter can also be formed in some instances. Simple terminals can also be formed, on which the catheters are positioned. The physician now introduces an ablation catheter, the catheter tip of which is formed from catoms and can be localized (e.g. by means of a position sensor) (FIG. 10c). When the physician reaches a point close to the ablation region, the hitherto unstructured catoms automatically form themselves into “bends”, which lead to the predefined ablation points P_AP (FIG. 10d). The instrument is thus formed optimally for the approach to each ablation point. It is possible here for all the points to be left automatically or for the approach to be initiated by the physician. The catoms then do not have to be “extracted” or remain unstructured in the body, as they are a fixed part of the instrument used.

In a further embodiment when forming guides the forms can also be based on variations of simple basic geometric forms instead of on a precise patient 3D model. For example in addition to a complex anatomically precise guide it is also possible to form simple “tubes” to pass through a branch.

Instead of a 3D data record it is also possible, in particular for applications in the heart, to use 4D data records (3D and time information, e.g. heart movement). The position of the instrument can then be determined correspondingly more precisely, e.g. by way of correlation with an ECG signal.

To summarize the abovementioned examples have the following common procedure:
Starting from an optionally segmented 3D data record of the target region to be treated (e.g. a vascular system) a 3D model is determined of the required aid (stent, coil, navigation or guidance aid, complete catheter or part of a catheter, e.g. catheter tip, etc.).
Depending on the model selected (stent, coil, guidance aid, catheter (tip) etc.) a corresponding “program” is used for the catoms in question and transferred to these.
The catoms are introduced unstructured, e.g. by way of a catheter, into the target region to be treated or they are part of a catheter or other instrument (see above) in unstructured form.
The units or catoms organize themselves in situ, e.g. after an appropriate “start command”, which can be previously determined, into a previously determined form (e.g. according to the 3D model) such as for example:
   one or more coils or stents,
   as a navigation or guidance aid,
   to form a complete catheter or
   to form a part of a catheter, e.g. a catheter tip, by means of a reforming operation.
After the end of the procedure, the temporary aids break down again. The catoms either remain in the body or are taken out again (e.g. by extraction).
The stationary aids (e.g. stents or coils) remain in a fixed form in the body, e.g. to close off the treated aneurysm permanently.

Two embodiments are possible in principle here:
The catoms themselves are “intelligent units”, which carry out their mutual localization and determine the next steps themselves, or
Some of their intelligence is “outsourced”, e.g. to an extracorporeal or (temporarily) intracorporeal “central computer”. The catoms can then only send this their respective position for example, the central computer manages the self-organization and calculates the next steps for each catom and then sends the corresponding commands back to the catoms, which carry out such commands accordingly. The catoms should then only be able, if necessary, to carry out certain navigations or reforming operations.

### REFERENCES

[1] “Magie des schlauen Sandes” (Magic of the smart sand), Der Spiegel, No. 6/2009 dated 02.02.2009.
[2] “Dynamic Physical Rendering”, Wikipedia, as at 10.03.2009
[3] Claytronics, CMU homepage of researcher group as at 10.03.2009
[4] “Distributed Localization of Modular Robot Ensembles”, Stanislav Funiak, Padmanabhan Pillai, Michael P. Ashley-Rollman, Jason D. Campbell, and Seth Copen Funiak, In Proceedings of Robotics: Science and Systems, June 2008 (and references therein).
[5] WO 2008/063473 A2.

1.-23. (canceled)

24. A method for forming an interventional aid with nanorobots consisting of catoms, comprising:
determining a faun of the interventional aid from a 3D image data record of a target region;
converting the determined form to a readable and executable program code for the catoms of the nanorobots;
transferring the determined form to a storage unit;
unstructured introducing the catoms to the target region; and
executing the program code for self-organizing the unstructured introduced catoms to form the interventional aid based on the determined form.

25. The method as claimed in claim 24, further comprising triggering the execution of the program code by a timer or a position sensor.

26. The method as claimed in claim 24, wherein the interventional aid is used in an endovascular target region.

27. The method as claimed in claim 24, wherein the interventional aid is a complete stent, and/or a complete coil, and/or a complete catheter, and/or a complete guide wire.

28. The method as claimed in claim 24, wherein the interventional aid is a part of a catheter, and/or a catheter tip, and/or a part of a guide wire, and/or a part of a guidance aid.

29. The method as claimed in claim 24, wherein the interventional aid is configured by temporarily self-organizing the unstructured introduced catoms into the determined form.

30. The method as claimed in claim 24, wherein the program code is executed to a degree on the catoms and/or to a further degree on an external computation unit communicated with the catoms.

31. The method as claimed in claim 24, wherein the target region is segmented from the 3D image data record based on marking points.

32. The method as claimed in claim 24, wherein the 3D image data record is supplemented by a time-related information to give a 4D image data record.

33. The method as claimed in claim 24, wherein the form of the catoms is broken down.

34. The method as claimed in claim 24, wherein the form is determined based on a three-dimensional model representing variants of a specific basic form.

35. The method as claimed in claim 24, wherein the form is determined based on a predetermined standard form.

36. A system unit for forming an interventional aid, comprising:
a computation device that determines a form of the interventional aid from a 3D image data record of a target region;
a plurality of nanorobots comprising catoms and a program code based on the determined form that communicate and exchange information with each other;
a storage unit that stores the determined form;
a device that unstructured introduces the catoms to the target region; and
a device that executes the program code to Ruin the interventional aid based on the determined form by self-organizing the unstructured introduced catoms.

37. The system unit as claimed in the claim 36, wherein the nanorobots is introduced into the target region by a catheter.

38. The system unit as claimed in the claim 36, further comprising a timer or a position sensor that triggers the execution of the program code.

39. The system unit as claimed in the claim 36, wherein the interventional aid is a complete stent, and/or a complete coil, and/or a complete catheter, and/or a complete guide wire.

40. The system unit as claimed in the claim 36, wherein the interventional aid is a part of a catheter, and/or a catheter tip, and/or a part of a guide wire, and/or a part of a guidance aid.

41. The system unit as claimed in the claim 36, wherein the program code is be executed to a degree on the catoms and/or to a further degree on an external computation unit communicated with the catoms.

42. The system unit as claimed in the claim 36, wherein the form is determined based on a three-dimensional model representing variants of a specific basic form.

43. The system unit as claimed in the claim 36, wherein the form is determined based on a predetermined standard form.
